(19) United States
(12) Patent Application Publication
(10) Pub. No.: US 2013/0004418 A1
(43) Pub. Date: Jan. 3, 2013

(54) INFILTRATION SOLUTION FOR TREATING AN ENAMEL LESION

(75) Inventors: Swen Neander, Hamburg (DE); Thomas Konig, Hamburg (DE); Stephan Neffgen, Pinneberg (DE); Sebastian Paris, Osdorf (DE); Hendrik Meyer-Luckel, Langwedel (DE)

(73) Assignee: ERNST MUHLBAUER GMBH & CO. KG, Norderfriedrichskoog (DE)

(21) Appl. No.: 13/579,820

(22) PCT Filed: Feb. 17, 2011
(86) PCT No.: PCT/EP11/52366

§ 371 Date: Aug. 17, 2012

(30) Foreign Application Priority Data
Feb. 17, 2010 (DE) 202010003032.3

(51) Int. Cl. A61K 51/00; A61Q 11/00

(52) U.S. Cl. 424/1.61

(57) ABSTRACT

The invention relates to an infiltration solution of a radiopaque metal compound for treating an enamel lesion, to a kit for dental application, and to the use thereof for preventing and/or treating (sealing) carious enamel lesions.

The invention relates to an infiltration solution of a radiopaque metal compound, to a kit for dental application, and to the use thereof for preventing and/or treating (sealing) carious enamel lesions. The kit comprises the infiltration solution of a radiopaque metal compound, according to the invention, as a first component, and a curable infiltrant, comprising polymerization or crosslinkable monomers, as a second component.

Carious enamel lesions here are essentially instances of carious damage that extend in the dental enamel but have not yet led to cavitation (formation of holes). Carious enamel lesions are demineralized regions of the dental enamel that may have a depth of up to 2-3 mm. The pore volume of a lesion body may amount to 5% to 25%.

WO 2007/131725 A1 has disclosed the treatment of carious enamel lesions by means of an infiltration method and infiltrants, to prevent cavitation and obviate the restoration with dental composites that is otherwise typically practiced. In the infiltration method, after any superficial remineralized layer present has been removed, the lesion is contacted with an infiltrant that is composed substantially of monomers, which then infiltrate. When the infiltrant has penetrated the lesion, the monomers are polymerized by means of photoactivation. This seals the lesion. The progression of the caries is halted.

Infiltration requires specific monomers or monomer mixtures, since known dental adhesives for dental composites (also known as bondings) are too slow and/or not sufficient in penetrating into the lesion and/or in fully penetrating (or infiltrating) the lesion. WO 2007/131725 A1 describes the use of monomers or monomer mixtures whereby the infiltrant has a penetration coefficient PC>50 cm/s.

A disadvantage of the infiltrants known from the prior art (e.g., WO 2007/131725 A1) is their inadequate radiopacity. They are substantially transparent (translucent) for X-rays and are therefore very difficult or impossible to recognize in a radiodiagnostic procedure. This robs of its value one of its most important instruments for recognizing the extent and the position of existing infiltrations. Apart from this, when using radiodiagnostics, it is difficult, owing to the inadequate radiopacity of the infiltrants, to determine any caries which may be progressing further beneath the infiltrated lesion, since any such caries is impossible or very hard to distinguish from the infiltrated region. In order to ascertain progressive caries, it is then necessary to take costly and inconvenient, precisely reproducible bitewing radiographs, of the kind described in German utility model application DE 202008006814 U1.

EP 2 153 812 A1 (not a prior publication) discloses the additional incorporation, into an infiltrant comprising crosslinking monomers, of radiopaque materials.

The object on which the invention is based is that of providing a possibility for further improving the radiopacity of those regions of a tooth that have been or are to be infiltrated.

This object is achieved by means of an infiltration solution according to claim 1 and also a kit composed of an infiltration solution of the invention and an infiltrant which comprises polymerizable or cross-linking monomers. Advantageous developments of the invention are specified in the dependent claims.

The invention has recognized that tooth regions restored by infiltration are not readily identifiable by means of radiodiagnostics and that possibly, through a greater radiopacity of the infiltrated lesion, it might be possible to produce, relative to the surrounding tooth and bone tissue, a level of contrast sufficient for radiodiagnostic investigation.

The inadequate contrasting as found for infiltrations does not arise in the case of the conventionally used dental materials of the kind employed in restoration or tooth replacement. The metallic restorations used in the prior art inherently generate a good contrast. The same applies to ceramic materials and polymeric composites, in which the pigments and/or fillers, added primarily for reasons of increasing the mechanical strength and reducing the contraction, provide a sufficient radiopaque contrast.

The center of the present invention is the provision of an infiltration solution which can be used in advance, prior to the implementation of the actual infiltration with crosslinking monomers, to introduce radiopaque materials into the lesion that increase the radiocontrast of the infiltrated region. The infiltration solution of the invention penetrates a lesion to at least approximately the same extent as an infiltrant which comprises crosslinking monomers. The volatile solvent evaporates or vaporizes, and so the radiopaque substances dissolved therein remain in the lesion and increase its radio contrast. If required, the infiltration solution of the invention can be applied two or more times in succession, in order to introduce more radiopaque materials into the treated lesion and so to increase its radiocontrast to the desired degree.

The radiopaque metal compound used in accordance with the invention is in solution in the solvent or solvent mixture. In accordance with the invention, this may be a true solution or else a colloidal solution.

For preparing a true solution it is possible, for example, to use organometallic compounds or metal salts which are soluble to the desired extent in the solvent in question.

For preparing a colloidal solution it is possible to use suitable nanoscale fillers. The term “colloidal solution” is used here with its definition as in Ullmann's Encyclopedia of Industrial Chemistry, 6th edition, volume 9, p. 31ff. In the case of colloidal solutions, therefore, nanoscale fillers with suitable radiocontrast, dispersed in the solution, may be present as the colloid. A solution is typically termed colloidal when the particle sizes of the colloidal particles are between 1 nm and 1 μm. The colloidal solution used in accordance with the invention may therefore more particularly be a colloidal dispersion of a nanoscale filler in a suitable (volatile) solvent. Preference in connection with the present invention is given to colloidal dispersions of particles having particle sizes of 1 to 100 nm. Particularly preferred are unaggregated or unagglomerated nanoscale fillers.

The present invention has recognized that it is possible to provide infiltration solutions comprising radiopaque nanoscale fillers and/or other radiopaque organic or inorganic metal compounds, more particularly salt-like metal compounds, for use as intended in the context of dental applications, when these solutions are present in the form of a true or colloidal solution.

Furthermore, the invention has recognized that the radiopaque contrast can be improved further if the infiltration solutions of nanoscale fillers and/or other organic or inorganic metal compounds, more particularly saltlike metal compounds, are introduced into the lesion separately from the infiltrant to be cured (optionally in two or more preceding steps).

In a first step, an infiltration solution comprising a volatile solvent and the dissolved radiopaque compound is employed, and in a second step an infiltrant to be cured that comprises substantially monomers and/or substantially monomers and a dissolved radiopaque metal compound is employed.

First of all a number of terms used in the context of the invention will be elucidated.

The term “infiltrant” identifies a liquid which is able to penetrate into a dental enamel lesion (a porous solid). An infiltrant comprises or consists of polymerizable and/or crosslinkable monomers. After penetrating a lesion, the infiltrant can be cured therein with polymerization and/or crosslinking of the monomers.

To be distinguished from an infiltrant of this kind is an infiltration solution of the invention, whose key constituents are solely volatile solvent and the metal compounds defined in claim 1. The application of an infiltration solution of the invention of this kind is therefore intended solely for transporting radiopaque materials into the lesion before the infiltrant itself is used. The solvent used as a vehicle for transporting the radiopaque materials into the lesion is subsequently evaporated or allowed to evaporate.

The penetration of a liquid (e.g., uncured resin (infiltrant) or infiltration solution, also referred to generally hereinafter as liquid resin) into a porous solid (dental enamel lesion) is described physically by the Washburn equation (equation 1, see below). In this equation it is assumed that the porous solid represents a bundle of open capillaries (Buckton G., Interfacial phenomena in drug delivery and targeting. Chur. 1995); in this case, the penetration of the liquid is driven by capillary forces.

$${d}^{2} = ( \frac{γ \cdot cos θ}{2 η} ) r \cdot t \qquad equation 1$$

d distance by which the liquid resin moves
γ surface tension of the liquid resin (with respect to air)
θ contact angle of a liquid resin (with respect to enamel)
η dynamic viscosity of the liquid resin
r capillary radius (pore radius)
t penetration time

The expression in parentheses in the Washburn equation is referred to as the penetration coefficient (PC, equation 2, see below) (Fan P. L. et al., Penetrativity of sealants. J. Dent. Res., 1975, 54: 262-264). The PC is composed of the surface tension of the liquid with respect to air (γ), the cosine of the contact angle of the liquid with respect to enamel (θ), and the dynamic viscosity of the liquid (η). The greater the value of the coefficient, the faster the penetration of the liquid into a given capillary or into a given porous bed. This means that a high value of PC can be obtained through high surface tension, low viscosities, and low contact angles, with the influence of the contact angle being comparatively small.

$$PC = ( \frac{γ \cdot cos θ}{2 η} ) \qquad equation 2$$

PC penetration coefficient
γ surface tension of the liquid resin (with respect to air)
θ contact angle of the liquid resin (with respect to enamel)
η dynamic viscosity of the liquid resin

Infiltration solutions of the present invention comprise radiopaque metal compounds in solution in solvent (true or colloidal solution).

The invention accordingly provides an infiltration solution for treating an enamel lesion, comprising:
(a) at least 25% by weight of a solvent or solvent mixture which is volatile at room temperature (23° C.), and
(b) in solution in the solvent or solvent mixture, a radiopaque metal compound having a radiopacity of more than 200% aluminum as determined in accordance with EN ISO 4049:2000.

A kit according to the invention for treating an enamel lesion comprises an infiltration solution of the invention and a curable infiltrant. The invention accordingly also provides a kit comprising an infiltration solution of the invention and a prior-art infiltrant. Suitable curable infiltrants are known from WO 2007/131725 A1, for example, the disclosure content of which is hereby, by reference, made part of the present application as well.

The invention is additionally realized by a method for infiltrating an enamel lesion, comprising the steps of:
(1) incorporating a radiopaque metal compound into the lesion by means of an infiltration solution of the invention, whose solvent is subsequently evaporated or left to evaporate,
(2) infiltrating the lesion, pretreated accordingly, with an infiltrant which comprises polymerizable and/or crosslinkable monomers,
(3) curing the infiltrant in the lesion.

The infiltration solution for use for step 1 has a high penetration coefficient PC and penetrates the lesion completely within a short time; the solvent is left to evaporate, and the radiopaque metal compound remains and is deposited in the lesion. Step (1) may if required be repeated a number of times in order to introduce into the lesion a sufficient amount of radiopaque metal compounds.

The infiltrant for use for step 2 may further comprise dissolved radiopaque compounds, as disclosed in EP 2 153 812 A1, for example. Serving as solvent in that case is the liquid resin or the crosslinking monomers.

In one variant of the invention, the infiltration solution of step (1) and the infiltrant of step (2) have the same or similar penetration coefficients. The effect of this is that the depth of penetration of the infiltration solution into the lesion in step (1) is the same as or similar to the depth of penetration of the infiltrant in step (2) then the lesion is subsequently infiltrated actually with crosslinkable monomers. This ensures that the labeling introduced, so to speak, in step (1) with radiopaque substances reaches to a similar depth as the actual infiltration with monomers that are subsequently cured, as carried out in step (2). In accordance with this variant, therefore, in the case of a kit according to the invention as well, the infiltration solution and the infiltrant have the same or similar penetration coefficients.

The PC value of the infiltration solution is preferably above 50 cm/s, with more preferred lower limits being 100, 200, and 300 cm/s. The upper limit attainable may be, for example, 1000 or 900 cm/s, dependent among other things on the solvent used. The infiltrant of the kits according to the invention may likewise have the stated minimum PC values, but in certain circumstances will have lower PC values than the infiltration solution, and so attainable upper limits of, for example, 600, 500, 400 or 300 cm/s may be present.

Through the infiltration solution of the invention and the method of the invention it is possible to increase by a multiple the amount of radiopaque compounds in infiltrated lesions, thereby making it possible for infiltrated lesions to be visualized more effectively by means of radiodiagnostics. The radiopacity of the infiltrated lesion body is preferably significantly greater than that of the (healthy) enamel. It is, however, also possible to adapt the radiopacity of the infiltrated lesion to that of the enamel, such that only lesional regions not infiltrated, and/or a further-progressing caries, would be detectable radiodiagnostically. More particularly, the provision of a relatively radiopaque infiltrated lesional region is intended to prevent a lesion treated with an infiltrant being wrongly diagnosed, in a subsequent radiographic investigation, as an active carious lesion, something which, in the case of treatment with a filling therapy, would result in an unnecessary loss of substance on the tooth. Any significant increase in the radiopacity of the lesion, wholly or partly compensating or even overcompensating for the mineral loss of the lesion relative to the undamaged enamel, is therefore desirable. The radiopacity of the lesion after treatment with the kit according to the invention is therefore preferably >100% Al, more preferably at least in the range of healthy enamel, and, very preferably, greater than that of the healthy enamel.

With the method of the invention, the amount and/or selection of the radiopaque compounds to be introduced is restricted to less of an extent than when a radiopaque compound is introduced only together with the infiltrant comprising liquid resins that is to be cured.

Suitable radiopaque metal compounds are soluble in the solvent of the infiltration solution.

The nanoscale fillers suitable in accordance with the invention are metal compounds, more particularly metal or mixed metal oxides, silicates, nitrides, sulfates, titanates, zirconates, stannates, tungstates or a mixture of these compounds. The term mixed metal oxide, nitride, etc., refers here to a chemical compound in which at least two metals and/or semimetals are bonded chemically to one another together with the corresponding (non)metal anion (oxide, nitride, etc.).

The nanoscale fillers which can be used in accordance with the invention are preferably zirconium dioxide, zinc oxide, tin dioxide, cerium oxide, silicon zinc oxides, silicon zirconium oxides, indium oxides and mixtures thereof with silicon dioxide and/or tin dioxide, strontium sulfate, barium sulfate, strontium titanate, barium titanate, sodium zirconate, potassium zirconate, magnesium zirconate, calcium zirconate, strontium zirconate, barium zirconate, sodium tungstate, potassium tungstate, magnesium tungstate, calcium tungstate, strontium tungstate and/or barium tungstate.

Nanoscale radiopaque fillers used with particular preference are selected from the group consisting of salts of the rare earth metals, of scandium, of yttrium, of barium and strontium, or tungstates. Suitable sparingly soluble salts are preferably sulfates, phosphates or fluorides.

Among the salts of the rare earth metals (elements 57-71), of scandium or of yttrium, the trifluorides are preferred. The preferred rare earth metals include lanthanum, cerium, samarium, gadolinium, dysprosium, erbium or ytterbium. Among their salts, preference is given to the fluorides, more particularly ytterbium trifluoride (YbF3). Preferred barium and strontium salts are fluorides, phosphates, and sulfates, more particularly the sulfates.

The expression “tungstate” encompasses metal compounds of the orthotungstates and polytungstates, the former being preferred.

The metal tungstate is preferably a tungstate compound of a polyvalent metal, more particularly of a divalent or trivalent metal. Suitable divalent metals include alkaline earth metals, such as magnesium, calcium, strontium or barium, more particularly calcium, strontium or barium. Strontium and barium tungstates are notable for particularly high radiopacity, since these compounds combine two good contrast agents with one another. Preferred trivalent metals include scandium, yttrium or rare earth metals, such as lanthanum, cerium, samarium, gadolinium, dysprosium, erbium or ytterbium. Here again, a particularly high radiopacity comes about from the fact that a good contrast agent (tungstate) is combined with a strongly contrast-forming metal. It is possible, furthermore, for the tungstates used in accordance with the invention (and/or the other nanoscale salts as well) to be doped with metal atoms. For this purpose the host lattice metal is preferably replaced by the dopant in an amount of up to 50 mol %, more preferably 0.1 to 40 mol %, even more preferably 0, 5 to 30 mol %, more particularly 1 to 25 mol %. The dopant selected may contribute to the radiopacity. For analytical reasons, however, it may also be of interest to select one or more doping metals which impart luminescent properties, more particularly photoluminescence. Dopants suitable for this purpose are known in the art and are often selected from a lanthanide different from the host lattice metal. Examples include combined doping with Eu and Bi, or the doping of Ce in combination with Nd, Dy or Tb, or Er in combination with Yb. It is equally possible to dope a tungstate as host lattice with a suitable lanthanide ion or another metal ion, e.g., Bi³⁺ or Ag⁺.

The nanoscale radiopaque fillers of the invention preferably have average particle sizes d₅₀ or regions of these particle sizes of less than 100 nm, preferably less than 25 nm, or between 1 nm and 80 nm, between 4 nm and 60 nm, between 6 nm and 50 nm, between 0.5 nm and 22 nm, between 1 nm and 20 nm, between 1 nm and 10 nm or between 1 nm and 5 nm.

Particular preference is given to unaggregated and unagglomerated nanoscale fillers present in isolation. Additionally preferred are fillers having a unimodal particle size distribution. The terms “aggregate” and “agglomerate” are used in the way in which they are defined in DIN 53206.

The nanoscale filler of the invention has a BET surface area (in accordance with DIN 66131 or DIN ISO 9277) of between 15 m²/g and 600 m²/g, preferably between 30 m²/g and 500 m²/g, and more preferably between 50 m²/g and 400 m²/g.

The nanoscale fillers are present in the form of colloidal or true solutions.

Suitable radiopaque metal compounds for true solutions are readily soluble (ionic) inorganic metal salts, such as halides and nitrates etc. Preference is given to fluorides, chlorides, iodides, and bromides. Preferred metals are those of the radiopaque fillers. Particularly suitable are, for example, cesium fluoride, rubidium fluoride, zinc bromide, etc.

Suitable radiopaque metal compounds of true solutions are also readily soluble (ionic) organic salts of carboxylic esters, etc. acetates or alkoxides, e.g., ethoxides, etc. It may be preferable for the organic salts to be polymerizable, such as acrylates and methacrylates (monomers). Examples of suitable monomers are zirconium acrylate, zirconyl dimethacrylate, zirconium tetra(meth)acrylate, zirconium carboxyethyl (meth)acrylate, zirconium(bromonorbornanelactonecarboxylate) tri(meth)acrylate, hafnium(meth)acrylate, hafnium carboxyethyl (meth)acrylate, strontium(meth)-acrylate, barium(meth)acrylate, ytterbium(meth)-acrylate, and yttrium(meth)acrylate.

Suitable radiopaque metal compounds are also covalent organometallic compounds such as triphenylbismuth compounds.

The dissolved metal compound is present at more than 5%, preferably more than 20%, more preferably more than 25%, very preferably at 30% to 65%, in the infiltration solution. These percentages are in percent by weight, unless otherwise defined.

The metal compound has a radiopacity as per the measurement procedure in the method according to DIN ISO 4049:2000 of preferably greater than 300% aluminum, preferably greater than 500% aluminum, most preferably greater than 500% aluminum.

Solvents or solvent mixtures suitable in accordance with the invention are those in which the radiopaque metal compounds are readily soluble or colloidally soluble.

With regard to step 1 of the method of the invention, suitable solvents or solvent mixtures are those which can be evaporated effectively from the lesion.

Preferred easy-evaporating solvents are protic or polar aprotic solvents. Suitable solvents have a vapor pressure at 20° C. of >10 hPa, preferably >about 20 hPa, more preferably >30 hPa. One particularly preferred range of vapor pressures for the solvent or for the preparation for infiltration lies between 30 to 300 hPa, more particularly 30 to 100 hPa. Particularly suitable solvents have a low molecular mass (<120 g/mol). The solvents are preferably selected from the group of the alcohols (preferably alkanols), ketones, ethers or esters. Suitable solvents are, for example, methanol, ethanol, 2-proponal, 1-propanol, butanol, 2-methyl-2-propanol, dimethyl ether, ethyl methyl ether, diethyl ether, tetrahydrofuran, propanone, butanone, ethyl acetate, and propyl acetate. The solvents may be used alone or as mixtures.

The solvents preferably have an evaporation index of less than 35 in accordance with DIN 53170:2009. Particularly preferred solvents have an evaporation index of less than 20, more preferably less than 10.

The solvent to be evaporated may function at the same time as a dryer for the lesion. A particularly preferred solvent is ethanol.

The infiltration solution may comprise customary dental or other additives such as initiators, accelerants, stabilizers, inhibitors, film-formers, dyes, fluorescent dyes, antibiotics, fluoridation agents, remineralizing agents, surfactants and also chelate complexing agents and/or crystallization inhibitors.

The infiltration solution may be part of a kit for the treatment of an enamel lesion. The kit comprises at least the infiltration solution of the invention and a curable infiltrant.

Suitable curable infiltrants are those of the prior art.

The kit may comprise an etchant. Suitable etchants are those of the prior art.

The kit may comprise an additional dryer.

The method of the invention may be preceded by an etching step in order to remove a superficial layer of the lesion. The etchant is removed and the lesion is dried.

The infiltration solution is applied, and infiltrates the lesion, and so the pore volume is completely cured. The solvent is allowed to evaporate. Evaporation may be assisted by measures such as air flow, supply of heat, etc.

The steps of the infiltration method, particularly step (1), may be repeated one or more times in order to achieve a further increase in the amount of radiopaque compounds in the lesion.

After a first and/or second infiltration, it is possible optionally to apply a lacquer or sealant which comprises fillers and which preferably has a penetration coefficient PC of below 50 cm/s, is compatible with the infiltrant, is cured together with the latter or separately, and produces a good bond. The sealant or lacquer preferably comprises the radiopaque nanoscale fillers of the invention, preferably at higher levels than does the infiltrant. Alternatively, the sealant or lacquer may also comprise other fillers, examples being barium- or strontium-containing inert dental glasses and/or ionomer glasses.

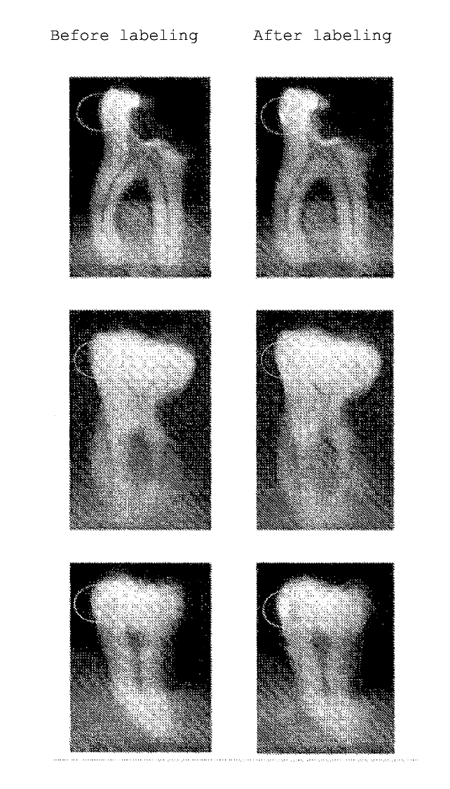

The invention is elucidated below by means of a number of examples. FIG. 1 shows X-ray photographs of natural lesions in molars before and after a treatment in accordance with the invention.

Substance abbreviations used and their meaning:

| | |
|---|---|
| TEDMA | Triethylene glycol dimethacrylate |
| UDMA | Urethane dimethacrylate (CAS 72869-86-4) |
| Bis-GMA | Bisphenol A glycidyl dimethacerylate (CAS 1565-94-2) |
| E3TMPTA | Ethoxylated trimethylolpropane triacrylate |
| CQ | Camphorquinone |
| EHA | Ethylhexyl p-N,N-dimethylaminobenzoate |
| BHT | 2,6-Di-tert-butylphenol |
| LTPO | Lucerin ® TPO (BASF) |

### Test Methods

### Surface Tension

The surface tension of the infiltrants was carried out by means of contour analysis on a hanging droplet (DSA 10, KRÜSS GmbH). The surface tension was measured on newly formed droplets over a time of 30 s, with one value being recorded about every 5 s. For this purpose the resins were delivered using a fine syringe and the droplet that formed was filmed with a digital camera. The surface tension was determined from the characteristic shape and size of the droplet, in accordance with the Young-Laplace equation. For each resin, three measurements were carried out in this way, and their average was reported as the surface tension.

### Density Determination

The densities of the infiltrants were determined using a pycnometer. For this determination, the density of air was deemed to be 0.0013 g/ml and the Earth's acceleration to be 9.8100 m/s².

### Contact Angle

Each individual measurement was carried out using enamel from bovine teeth. For this purpose, bovine teeth were embedded in a synthetic resin and the enamel surface was wet-polished using a sanding machine (Struers GmbH) with abrasive papers (80, 500, and 1200 grades), thus providing planer enamel surfaces approximately 0.5×1.0 cm in size for the contact angle measurements. Up until the time of measurement, the enamel samples were stored in distilled water, and prior to measurement they were dried with ethanol and compressed air.

The contact angle was measured using a video contact angle measuring instrument (DSA, KRUSS GmbH). In this measurement, a drop of the infiltrant was applied to the enamel surface using a microliter syringe, and within a period of 10 s, up to 40 individual pictures of the droplet were taken, under computer control, and the contact angle was determined by means of droplet contour analysis software.

### Dynamic Viscosity

The viscosity of the resins was measured at 23° C. using a dynamic plate/plate viscometer (Dynamic Stress Rheometer, Rheometric Scientific Inc.). Measurement took place in Steady Stress Sweep mode with slot sizes of 0.1 to 0.5 mm in the shear stress range from 0 to 50 Pa, without preliminary shearing of the resins.

### Radiopacity

The measurement of the radiopacity took place by irradiation of specimens approximately 1 mm thick in accordance with the provisions of EN ISO 4049:2000 (Polymer-based filling, restorative and luting materials). For determining the radiopacity of materials from which it is not possible per se to produce specimens by curing, curable composites were produced. From these it was possible The radiopacity of the radiopaque compound (100%) was determined from a plot of the weight fraction of radiopaque compound in the test specimen/composite against the measured radiopacity, by extrapolation to 100% radiopaque compound.

### EXAMPLE 1

First of all, the radiopacity of triphenylbismuth (Ph3Bi) and also of the following salts was ascertained: cesium fluoride (CsF), cesium iodide (CsI), barium fluoride (BaF2), strontium fluoride (SrF2), ytterbium fluoride (YbF3), yttrium fluoride (YF3), strontium chloride (SrCl2), zinc bromide (ZnBr2), and rubidium fluoride (RbF).

The stated salts were each homogenized at 20, 40, and percent by weight in photocuring resin (40% by weight Bis-GMA, 20% by weight UDMA, 20% by weight TEDMA, 20% by weight of ethoxylated Bis-GMA, 0.2% by weight CQ, 0.2% by weight EHA, 0.2% by weight LTPO, and 0.005% by weight BHT) together with 2% of Aerosil® (R812S). For this homogenization, all of the components were mixed twice at 3000 rpm with a SpeedMixer (from Hauschild). The pastes were subsequently dispersed by a triple-roll mill and were mixed with the SpeedMixer again at 3000 rpm for 20 s. Any air bubbles present were removed by brief degassing of the paste in a desiccator.

Directly after preparation of the pastes/composites, specimens 1 mm thick in accordance with EN ISO 4049:2000 were produced by exposure to light. The exact thickness of the specimens was ascertained using a gauge. The specimens were placed together with an aluminum stepwedge (purity>98% aluminum, with less than 0.1% copper fraction and less than 1% iron fraction) on an X-ray film (Ultraspeed DF-50 dental film, film sensitivity D, from Kodak). Specimen, aluminum stepwedge, and film were irradiated with X-rays with an acceleration voltage of 65 kV using an analog single-phase X-ray instrument from Gendex, from a distance of 400 mm for 0.4 s. After the film had been developed and fixed, the degrees of blackening of the image of the specimens and of the aluminum stepwedge were measured, a blackening plot (degree of blackening against aluminum step height) for the aluminum step-wedge was plotted, and the values of the radiopacities for each specimen were determined from the graph.

In addition, the radiopacity of the photocuring composite without (0% by weight) radiopaque salt was measured in the same way. The composite consisted only of the photocuring resin and 2% of Aerosil (R812S).

The radiopacity specimens of hafnium carboxyethylacrylate were produced by curing a 60% strength alcoholic solution. The solution additionally contained photoinitiators (1 part by weight CQ, 1.6 parts by weight EHA), dissolved at room temperature by magnetic stirrer over the course of an hour. Following introduction into the mold, the solvent was evaporated in order to give a test specimen suitable for measurement.

The radiopacities [in % aluminum] found for the composites produced were as follows:

| % by weight | YbF₃ | CsI | RbF | SrF₂ | ZnCl₂ | BaF₂ | Ph₃Bi | CsF | SrCl₂ | YF₃ | Hafnium carboxyethylacrylate |
|---|---|---|---|---|---|---|---|---|---|---|---|
| 0 | 19 | 19 | 19 | 19 | 19 | 19 | 19 | 19 | 19 | 19 | |
| 20 | 99 | 87 | 130 | 110 | 128 | 88 | 145 | 85 | 98 | 85 | |
| 40 | 287 | 266 | 329 | 260 | — | 228 | 252 | 210 | 228 | 167 | |
| 60 | 530 | 489 | 484 | 468 | 450 | 449 | 446 | 434 | 422 | 316 | |
| Extrapolation | >700 | >700 | >700 | >700 | >700 | >700 | >700 | >700 | >700 | >500 | |
| to 100% by | | | | | | | | | | | |
| weight | | | | | | | | | | | |
| 100 | | | | | | | | | | | 257 |

In addition, the radiopacity of healthy human enamel is measured in accordance with the radiopacity determination method described above. It amounted to 158% aluminum. This value is in good agreement with the figures in the relevant literature for human enamel, of around 160% Al.

### EXAMPLE 2

A 40% by weight CsF containing ethanolic solution (infiltration solution) and also a 15% by weight CsF containing polymerizable solution (curable infiltrant; 15% CsF, 67.5% TEDMA, 16.9% E3TMPTA, 0.2% EHA, 0.2% CQ, 0.2% LTPO, and 0.005% BHT) were prepared. After a short stirring time, the solutions were clear.

The penetration coefficient of the ethanolic 40% strength CsF infiltration solution was about 900 cm/s. The evaporation index in accordance with DIN 53170:2009 was 8.

The penetration coefficient of the curable 15% strength CsF infiltrant was about 150 cm/s.

### EXAMPLE 3

### Evaporation Experiments

For a further assessment of the suitability of solvent for evaporating very rapidly and completely from enamel lesions, the degree of evaporation [%] was determined in a first experiment at 37° C. (mouth temperature). In this experiment, 0.1 g of each solvent was left to evaporate at room temperature in a crystallizing dish (d=40 mm). The weight loss was monitored as a function of the time [s] on an analytical balance and was expressed in relation to the initial quantity. The quantity of solvent used corresponds approximately to the quantity to be used for an infiltration treatments.

In a second experiment (at 23° C.), the solvent was additionally blown in a stream of air at a pressure of 2 bar. The degrees of evaporation [%] by blowing, with the higher mouth temperature, would then have to be even higher than the values shown here.

TABLE
Degrees of evaporation:

| | | 60 s | 120 s | 180 s |
|---|---|---|---|---|
| Ethanol | 37° C. | | >90% | 100% |
| | Stream of air | >80% | | |
| Propanol | 37° C. | | 80 | |
| | Stream of air | >90% | | |

### EXAMPLE 4

In the example below, natural approximal lesions in three molars (human teeth) were treated.

The infiltration solution used was a solution of 21% by weight CsF in ethanol.

A polymerizable infiltrant was produced in accordance with EP 2145613 A1, table resin 2, in which 0.5% by weight CQ, 0.84% by weight EHA, and 0.002% by weight BHT were dissolved by stirring under yellow-light conditions. Following its production, the infiltrant was stored in the absence of light prior to use.

Implementation of the method of the invention:

The approximal lesion was contacted with the infiltration solution for 30 s. The lesion was then exposed to a stream of oil-free air for 30 s. The treatment was repeated three times.

Next, twice in succession, an infiltration was carried out with polymerizable infiltrant. The period of application of the infiltrant at the first infiltration was 3 min, and at the second infiltration 1 min. After each infiltration step, the infiltrated lesion was exposed for 40 s each time to blue light from an LED polymerization lamp. This led to the reliable curing of the infiltrant.

Standardized X-ray photographs were taken of the molars before and after implementation of the method of the invention (conventional F film, 70 kV, 0.4 s). The photographs are shown in FIG. 1.

With all three molars, an approximal carious lesion can be seen in the form of radiological lightening (marked with a circle in FIG. 1) prior to the treatment in accordance with the invention. After the treatment in accordance with the invention, a marked increase in the radiopacity of all three lesions is apparent (FIG. 1). In this case, the radiodensity of the treated lesions was above that of the surrounding enamel.

1. An infiltration solution for treating an enamel lesion, comprising:
(a) at least 25% by weight of a solvent or solvent mixture which is volatile at room temperature (23° C.), and
(b) in solution in the solvent or solvent mixture, a radiopaque metal compound having a radiopacity of more than 200% aluminum as determined in accordance with EN ISO 4049:2000.

2. The infiltration solution of claim 1, wherein the infiltration solution at room temperature (23° C.) has a penetration coefficient PC of greater than 100 cm/s.

3-15. (canceled)

16. The infiltration solution of claim 1, wherein said infiltration solution at room temperature (23° C.) has a penetration coefficient PC of greater than 200 cm/s.

17. The infiltration solution of claim 1, wherein said infiltration solution at room temperature (23° C.) has a penetration coefficient PC of greater than 300 cm/s.

18. The infiltration solution of claim 1, wherein said solvent or solvent mixture (a) has an evaporation index EI of less than 35 according to DIN 53170:2009.

19. The infiltration solution of claim 1, wherein said solvent or solvent mixture (a) has an evaporation index EI of less than 10 according to DIN 53170:2009.

20. The infiltration solution of claim 1, wherein said infiltration solution is composed to an extent of at least 80% by weight of the solvent or solvent mixture (a) and of the dissolved metal compound (b).

21. The infiltration solution of claim 1, wherein said infiltration solution is composed to an extent of at least 90% by weight of the solvent or solvent mixture (a) and of the dissolved metal compound (b).

22. The infiltration solution of claim 1, wherein said infiltration solution is composed to an extent of at least 95% by weight of the solvent or solvent mixture (a) and of the dissolved metal compound (b).

23. The infiltration solution of claim 1, wherein said solvent or solvent mixture (a) is selected from the group consisting of alcohols, ethers, ketones, and esters.

24. The infiltration solution of claim 1, wherein said infiltration solution comprises at least 30% by weight of the solvent or solvent mixture (a).

25. The infiltration solution of claim 1, wherein said infiltration solution comprises at least 35% to 70% by weight, of the solvent or solvent mixture (a).

26. The infiltration solution of claim 1, wherein said dissolved metal compound (b) has a radiopacity of greater than 300% aluminum.

27. The infiltration solution of claim 1, wherein said dissolved metal compound (b) has a radiopacity of greater than 500% aluminum.

28. The infiltration solution of claim 1, wherein said dissolved metal compound (b) has a radiopacity of greater than 700% aluminum.

29. The infiltration solution of claim 1, wherein said dissolved metal compound (b) is the compound of a metal with an atomic number of 30 or higher.

30. The infiltration solution of claim 1, wherein said dissolved metal compound (b) is the compound of a metal with an atomic number of 38 to 83.

31. The infiltration solution of claim 1, wherein said dissolved metal compound (b) is a salt.

32. The infiltration solution of claim 1, wherein said dissolved metal compound is an organometallic compound.

33. The infiltration solution of claim 1, wherein said infiltration solution comprises at least 5% by weight of the dissolved metal compound.

34. The infiltration solution of claim 1, wherein said infiltration solution comprises at least 20% by weight of the dissolved metal compound.

35. The infiltration solution of claim 1, wherein said infiltration solution comprises at least 25% by weight of the dissolved metal compound.

36. The infiltration solution of claim 1, wherein said infiltration solution comprises at least 30 to 65% by weight of the dissolved metal compound.

37. The infiltration solution of claim 1, wherein said infiltration solution comprises the dissolved metal compound in colloidal solution.

38. The infiltration solution of claim 1, wherein said infiltration solution comprises the dissolved metal compound in true solution.

39. A kit for treating an enamel lesion, comprising the infiltration solution of claim 1 and a curable infiltrant.

40. The kit claim 39, wherein said enamel lesion is a carious enamel lesion.
